(19) United States
(12) Patent Application Publication
(10) Pub. No.: US 2011/0237675 A1
(43) Pub. Date: Sep. 29, 2011

(54) PHARMACEUTICAL FORMULATIONS

(75) Inventors: Yi Gao, Vernon Hills, IL (US); Tzuchi R. Ju, Vernon Hills, IL (US); Dennis Y. Lee, Highland Park, IL (US); Nicole Nguyen, Winfield, IL (US); Huailiang Wu, Long Grove, IL (US)

(73) Assignee: Abbott Laboratories, Abbott Park, IL (US)

(21) Appl. No.: 13/151,555

(22) Filed: Jun. 2, 2011

Related U.S. Application Data
(63) Continuation of application No. 11/871,514, filed on Oct. 12, 2007.
Application No. 11/871,514 is a continuation-in-part of application No. 11/548,982, filed on Oct. 12, 2006, now abandoned.
Application No. 11/548,982 is a continuation-in-part of application No. 11/399,983, filed on Apr. 7, 2006, now abandoned.
Application No. 11/871,514 is a continuation-in-part of application No. 11/548,960, filed on Oct. 12, 2006, now abandoned.
Application No. 11/548,960 is a continuation-in-part of application No. 11/399,964, filed on Apr. 7, 2006, now abandoned.
Application No. 11/871,514 is a continuation-in-part of application No. 11/549,005, filed on Oct. 12, 2006, now abandoned.
Application No. 11/549,005 is a continuation-in-part of application No. 11/400,113, filed on Apr. 7, 2006, now abandoned.
(60) Provisional application No. 60/829,255, filed on Oct. 12, 2006.
(60) Provisional application No. 60/669,699, filed on Apr. 8, 2005.
(60) Provisional application No. 60/669,699, filed on Apr. 8, 2005.
(60) Provisional application No. 60/669,699, filed on Apr. 8, 2005.

(51) Int. Cl. A61K 31/192; A61P 3/06; A61P 3/00

(52) U.S. Cl. 514/571

(57) ABSTRACT

The present invention provides a modified release formulation comprising an active agent in a hydrophilic polymer matrix wherein the active agent is a salt of fenofibric acid wherein the release rate of the formulation in an in vitro dissolution is substantially independent of the ionic strength of the dissolution media.

### RELATED APPLICATION INFORMATION

This application claims priority to U.S. Application No. 60/829,255, filed Oct. 12, 2006, the contents of which are herein incorporated by reference.

This application is a continuation-in-part of U.S. application Ser. No. 11/548,960, filed on Oct. 12, 2006, which is a continuation-in-part of U.S. application Ser. No. 11/399,964, filed on Apr. 7, 2006, which claims priority to U.S. Application No. 60/669,699, filed on Apr. 8, 2005, the contents of each of which are herein incorporated by reference.

This application is a continuation-in-part of U.S. application Ser. No. 11/548,982, filed on Oct. 12, 2006, which is a continuation-in-part of U.S. application Ser. No. 11/399,983, filed on Apr. 7, 2006, which claims priority to U.S. Application No. 60/669,699, filed on Apr. 8, 2005, the contents of each of which are herein incorporated by reference.

This application is a continuation-in-part of U.S. application Ser. No. 11/549,005, filed on Oct. 12, 2006, which is a continuation-in-part of U.S. application Ser. No. 11/400,113, filed on Apr. 7, 2006, which claims priority to U.S. Application No. 60/669,699, filed on Apr. 8, 2005, the contents of each of which are herein incorporated by reference.

### FIELD OF THE INVENTION

The present invention relates to solid dosage forms comprising salts of 2-[4-(4-chlorobenzoyl)phenoxy]-2-methyl-propanoic acid.

### BACKGROUND OF THE INVENTION

2-[4-(4-chlorobenzoyl)phenoxy]-2-methyl-propanoic acid,1-methylethyl ester, also known as “fenofibrate”, from the family of fibrates, is a lipid-regulating agent. Fenofibrate is described in, for example, U.S. Pat. Nos. 3,907,792, 4,895,726, 6,074,670 and 6,277,405. Fenofibrate is commercially available in a variety of different formulations and is used in the treatment of adult endogenous hyperlipidemias, hypercholesterolemias and hypertriglyceridemias. The active metabolite of fenofibrate is 2-[4-(4-chlorobenzoyl)phenoxy]-2-methyl-propanoic acid, which is also known as fenofibric acid.

One of the challenges associated with fibrates, such as fenofibrate, is that these compounds are hydrophobic and poorly soluble in water. Thus, the bioavailability of these compounds (i.e., their absorption in the digestive tract) can be low. Due to the hydrophobic nature and poor solubility of fenofibrate in water, absorption of fenofibrate in the digestive tract of a subject is increased after ingestion of food by the subject (when compared to when the subject ingests the fenofibrate under fasting conditions). This food effect is undesirable when comparing the bioavailability of fenofibrate in fed versus fasting conditions. Additionally, subject compliance is an issue with drugs having a food effect because the patient must coordinate administration of the drug with the ingestion of food. Recently, complex technologies have been used to overcome the food effect issues associated with fenofibrate.

In contrast to fenofibrate, fenofibric acid has higher solubility in the small intestine region. However, this enhanced solubility could cause problems in connection with controlling the delivery of fenofibric acid (such as, the potential for the Cₘₐₓ to exceed the accepted (approved) limits of a reference pharmaceutical composition containing fenofibrate). For example, immediate release dosage forms comprising amorphous fenofibric acid are described, for example, in U.S. Patent Application No. 2005/0148594. As reported therein, the formulations comprising amorphous fenofibric acid when administered to a subject, exhibit a bioavailability that is twice as high as a fenofibrate-containing capsule formulation described in Example 6 of said published application. Thereupon, in view of aforementioned described difference in solubility, the active ingredient, namely, fenofibrate, simply cannot be replaced with fenofibric acid in such dosage forms.

Moreover, there is a need in the art for solid dosage forms of fenofibric acid that exhibit a lack of a significant food effect when administered to a patient under fed or fasted conditions. Such solid dosage forms would improve patient compliance by giving the patient the flexibility to take said solid dosage form under either fed or fasted conditions.

The release rate of a robust drug formulation will be substantially independent of properties of the dissolution media. For example, a robust formulation will have essentially the same release rates in dissolution media of differing ionic strengths. In humans, normal fasting levels for the ionic strength in the GI tract is 0.10-0.14 and higher values are induced by the intake of food. It therefore follows that one would expect that the release rate of a robust drug formulation will exhibit minimal variation under fed and fasted conditions in the GI tract. A further feature of a robust drug formulation is that its release rate will not be effected during rigorous steps in scaled-up of manufacturing processes.

It is the object of the present invention to provide modified release fenofibric acid formulations which are robust. Consequentially the release rate of the formulations of the present invention are substantially independent of the ion-strength of dissolution medium. This object is achieved, according to the present invention, by a hydrophilic gel forming matrix formulation having a prolonged release of fenofibric acid upon exposure to the dissolution media, characterized in that the release rate is substantially ionic-strength independent.

Applicants have found several factors contribute in making a modified release fenofibric acid formulations robust. One factor is the salt selection. Applicants have discovered that robust fenofibric acid formulations should comprise a soluble salt. Second, the percentage of the fenofibric acid salt in the formulation also impacts the robustness of the formulation. Finally, the presence or absence of a drug enteric coating may have some influence on the robustness of the formulation.

### SUMMARY OF THE INVENTION

In one aspect, the present invention provides a modified release formulation comprising an active agent in a hydrophilic polymer matrix wherein the active agent is a salt of fenofibric acid wherein the release rate of the formulation in an in vitro dissolution is substantially independent of the ionic strength of the dissolution media.

### BRIEF DESCRIPTION OF THE FIGURES

FIG. 1 shows the IDR values of seven salts of fenofibric acid and fenofibric acid verses the difference in drug release at 8 hours in an in vitro dissolution at high and low ionic strengths.

FIG. 2 shows the in vitro dissolution profile of fenofibric acid tablets when done in dissolution media of 0.05M and 0.3M.

FIG. 3 shows the in vitro dissolution profile of fenofibric acid choline salt tablets when done in dissolution media of 0.05M and 0.3M.

FIG. 4 shows the in vitro dissolution profile of fenofibric acid metformin salt tablets when done in dissolution media of 0.05M and 0.3M.

FIG. 5 shows the in vitro dissolution profile of fenofibric acid procaine salt tablets when done in dissolution media of 0.05M and 0.3M.

FIG. 6 shows the in vitro dissolution profile of fenofibric acid diethanolamine salt tablets when done in dissolution media of 0.05M and 0.3M.

FIG. 7 shows the in vitro dissolution profile of fenofibric acid ethanolamine salt tablets when done in dissolution media of 0.05M and 0.3M.

FIG. 8 shows the in vitro dissolution profile of fenofibric acid calcium salt tablets when done in dissolution media of 0.05M and 0.3M.

FIG. 9 shows the in vitro dissolution profile of fenofibric acid tris salt tablets when done in dissolution media of 0.05M and 0.3M.

FIG. 10 shows the in vitro dissolution profiles of fenofibric acid tablets and fenofibric acid choline salt tablets at 32.5% drug load when done in dissolution media of 0.05M and 0.3M.

FIG. 11 shows the in vitro dissolution profiles of fenofibric acid tablets and fenofibric acid choline salt tablets at 65.5% drug load when done in dissolution media of 0.05M and 0.3M.

FIG. 12 shows the in vitro dissolution profiles of coated and uncoated fenofibric acid choline salt tablets when done in dissolution media of 0.05M and 0.3M.

### DETAILED DESCRIPTION

Another aspect of the present invention provides a modified release formulation comprising an active agent in a hydrophilic polymer matrix wherein the active agent is a salt of fenofibric acid and wherein the solubility of the active agent is greater than 16.1 mg/ml in water.

Another aspect of the present invention provides a modified release formulation comprising an active agent in a hydrophilic polymer matrix wherein the active agent is a salt of fenofibric acid and wherein the solubility of the active agent is at least 19.0 mg/ml in water.

In one aspect, the present invention relates to a modified release formulation comprising an active agent in a hydrophilic polymer matrix wherein the active agent is a salt of fenofibric acid and the salt is selected from the group consisting of choline, ethanolamine, and diethanolamine, and wherein the solubility of the active agent is greater than 16.1 mg/ml in water.

Another aspect of the present invention provides a modified release formulation comprising an active agent in a HPMC matrix wherein the active agent is a salt of fenofibric acid and wherein the solubility of the active agent is greater than 16.1 mg/ml in water.

Another aspect of the present invention provides a modified release formulation comprising an active agent in a HPMC matrix wherein the active agent is a salt of fenofibric acid and wherein the solubility of the active agent is at least 19.0 mg/ml in water.

Another aspect of the present invention provides a modified release formulation comprising an active agent in a hydrophilic polymer matrix wherein the active agent is a salt of fenofibric acid and wherein the IDR of the active agent is greater than 7.09 mg/min/cm² at a pH of 6.8.

Another aspect of the present invention provides a modified release formulation comprising an active agent in a hydrophilic polymer matrix wherein the active agent is a salt of fenofibric acid and the salt is selected from the group consisting of cholinc, ethanolamine, and diethanolamine, and wherein the IDR of the active agent is greater than 7.09 mg/min/cm² at a pH of 6.8.

Another aspect of the present invention provides a modified release formulation comprising an active agent in a HPMC matrix wherein the active agent is a salt of fenofibric acid and wherein the IDR of the active agent is greater than 7.09 mg/min/cm² at a pH of 6.8.

Another aspect of the present invention provides a modified release formulation comprising an active agent in a hydrophilic polymer matrix wherein the active agent is a salt of fenofibric acid and wherein the IDR of the active agent is at least 8.05 mg/min/cm² at a pH of 6.8.

Another aspect of the present invention provides a modified release formulation comprising an active agent in a HPMC matrix wherein the active agent is a salt of fenofibric acid and wherein the IDR of the active agent is at least 8.05 mg/min/cm² at a pH of 6.8.

Another aspect of the present invention provides a modified release formulation comprising an active agent in a hydrophilic polymer matrix wherein the active agent is a salt of fenofibric acid wherein the release rate of the formulation in an in vitro dissolution is substantially independent of the ionic strength of the dissolution media.

Another aspect of the present invention provides a modified release formulation comprising an active agent in a HPMC matrix wherein the active agent is a salt of fenofibric acid wherein the release rate of the formulation in an in vitro dissolution is substantially independent of the ionic strength of the dissolution media.

Another aspect of the present invention provides a modified release formulation comprising an active agent in a hydrophilic polymer matrix wherein the active agent is a salt of fenofibric acid and wherein in an in vitro dissolution the difference in percentage dissolved at time points 0.5, 1, 2, 4, 6, and 8 hours is not greater than 25% when dissolved in dissolution media of 0.05M and 0.3M.

Another aspect of the present invention provides a modified release formulation comprising an active agent in a hydrophilic polymer matrix wherein the active agent is a salt of fenofibric acid and the salt is selected from the group consisting of choline, ethanolamine, and diethanolamine, and wherein in an in vitro dissolution the difference in percentage dissolved at time points 0.5, 1, 2, 4, 6, and 8 hours is not greater than 25% when dissolved in dissolution media of 0.05M and 0.3M.

Another aspect of the present invention provides a modified release formulation comprising an active agent in a HPMC matrix wherein the active agent is a salt of fenofibric acid and wherein in an in vitro dissolution the difference in percentage dissolved at time points 0.5, 1, 2, 4, 6, and 8 hours is not greater than 25% when dissolved in dissolution media of 0.05M and 0.3M.

Another aspect of the present invention provides a modified release formulation comprising an active agent in a hydrophilic polymer matrix wherein the active agent is a salt of fenofibric acid and wherein in an in vitro dissolution the difference in percentage dissolved at time points 0.5, 1, 2, 4, 6, and 8 hours is not greater than 21.4% when dissolved in dissolution media of 0.05M and 0.3M.

Another aspect of the present invention provides a modified release formulation comprising an active agent in a HPMC matrix wherein the active agent is a salt of fenofibric acid and wherein in an in vitro dissolution the difference in percentage dissolved at time points 0.5, 1, 2, 4, 6, and 8 hours is not greater than 21.4% when dissolved in dissolution media of 0.05M and 0.3M.

Another aspect of the present invention provides a modified release formulation comprising an active agent in a hydrophilic polymer matrix wherein the active agent is a salt of fenofibric acid wherein the percentage of active agent in the formulation is between 33% and 75%.

Another aspect of the present invention provides a modified release formulation comprising an active agent in a HPMC matrix wherein the active agent is a salt of fenofibric acid wherein the percentage of active agent in the formulation is between 33% and 75%.

Another aspect of the present invention provides a modified release formulation comprising an active agent in a hydrophilic polymer matrix wherein the active agent is a salt of fenofibric acid wherein the percentage of active agent in the formulation is between 50% and 75%.

Another aspect of the present invention provides a modified release formulation comprising an active agent in a hydrophilic polymer matrix wherein the active agent is a salt of fenofibric acid and the salt is selected from the group consisting of choline, ethanolamine, and diethanolamine, wherein the percentage of active agent in the formulation is between 50% and 75%.

Another aspect of the present invention provides a modified release formulation comprising an active agent in a HPMC matrix wherein the active agent is a salt of fenofibric acid wherein the percentage of active agent in the formulation is between 50% and 75%.

Another aspect of the present invention provides a modified release formulation comprising an active agent in a hydrophilic polymer matrix wherein the active agent is a soluble salt of fenofibric acid wherein the percentage of active agent in the formulation is between 33% and 75%.

Another aspect of the present invention provides a modified release formulation comprising an active agent in a hydrophilic polymer matrix wherein the active agent is a soluble salt of fenofibric acid wherein the percentage of active agent in the formulation is between 50% and 75%.

Another aspect of the present invention provides a modified release formulation comprising an active agent in a hydrophilic polymer matrix wherein the active agent is a salt of fenofibric acid wherein the percentage of active agent in the formulation is between 33% and 75% and wherein the release rate of the formulation is substantially independent of the ionic strength of the dissolution media.

Another aspect of the present invention provides a modified release formulation comprising an active agent in a hydrophilic polymer matrix wherein the active agent is a salt of fenofibric acid wherein the percentage of active agent in the formulation is between 50% and 75% and wherein the release rate of the formulation is substantially independent of the ionic strength of the dissolution media.

Another aspect of the present invention provides a hydrophilic polymer matrix wherein the active agent is a salt of fenofibric acid wherein the difference in disintegration times of the active agent when disintegrated in media of 0.3M or 0.05M ionic strength is less than 475 minutes.

Another aspect of the present invention provides a hydrophilic polymer matrix wherein the active agent is a salt of fenofibric acid and the salt is selected from the group consisting of choline, ethanolamine, and diethanolamine, wherein the difference in disintegration times of the active agent when disintegrated in media of 0.3M or 0.05M ionic strength is less than 475 minutes.

Another aspect of the present invention provides a hydrophilic polymer matrix wherein the active agent is a salt of fenofibric acid wherein the difference in disintegration times of the active agent when disintegrated in media of 0.3M or 0.05M ionic strength is less than 100 minutes.

Another aspect of the present invention provides a HPMC matrix wherein the active agent is a salt of fenofibric acid wherein the difference in disintegration times of the active agent when disintegrated in media of 0.3M or 0.05M ionic strength is less than 475 minutes.

Another aspect of the present invention provides a HPMC matrix wherein the active agent is a salt of fenofibric acid wherein the difference in disintegration times of the active agent when disintegrated in media of 0.3M or 0.05M ionic strength is less than 100 minutes.

### DEFINITIONS

As used in this specification and the appended claims, the singular forms “a,” “an” and “the” include plural references unless the context clearly dictates otherwise. Thus, for example, reference to “an active agent” includes a single active agent as well two or more different active agents in combination, reference to “an excipient” includes mixtures of two or more excipients as well as a single excipient, and the like.

In describing and claiming the present invention, the following terminology will be used in accordance with the definitions set out below.

As used herein, the term “about” is used synonymously with the term “approximately.” Illustratively, the use of the term “about” indicates that values slightly outside the cited values, namely, plus or minus 10%. Such dosages are thus encompassed by the scope of the claims reciting the terms “about” and “approximately.”

As used herein, the terms “active agent,” “pharmacologically active agent,” and “drug” are used interchangeably herein to refer to salts of 2-[4-(4-chlorobenzoyl)phenoxy]-2-methyl-propanoic acid (fenofibric acid). The terms also encompass buffered 2-[4-(4-chlorobenzoyl)phenoxy]-2-methyl-propanoic acid. Salts of fenofibric acid include, but are not limited to choline, ethanolamine, diethanolamine, dicyclohexylamine, tromethamine, lysine, piperazine, calcium, cyclohexylamine, procaine, metoformin, potassium, lysine, meglumine, diethylamine, sodium and ethylenediamine. Examples of counter-ions that can be used to provide buffered fenofibric acid, include, but are not limited to, calcium hydroxide, choline hydroxide, diethylethanolamine, diethanolamine, ethylenediamine, guanidine, magnesium hydroxide, meglumine, ethanolamine, piperazine, peperidine, sodium hydroxide, triethylamine, tromethamine, benzathine, benzene-ethanamine, adenine, aluminum hydroxide, ammonium hydroxide, cytosine, diethylamine, glucosamine, guanine, nicotinamide, potassium hydroxide, zinc hydroxide, hydrabamine, tributylamine, deanol, epolamine, lithium hydroxide, procaine, pyridoxine, triethanolamine, ornithine, glycine, lysine, arginine, valine, serine, proline, aspartic acid, alanine, isoleucine, leucine, methionine or threnine. The solid state form of the active agent used in preparing the solid dosage forms of the present invention is not critical. For example, active agent used in preparing the solid dosage form can be amorphous or crystalline. The final dosage form contains at least a detectable amount of crystalline active agent. The crystalline nature of the active agent can be detected using powder X-ray diffraction analysis, by differential scanning calorimetry or any other techniques known in the art.

As used herein, the term “cloud point” refers to a phenomenon observed in HPMC gels with increase in their temperature resulting in a precipitation of the polymer molecules, a property which can be measured by light transmission. The temperature at which light transmission reaches 50% is called cloud point.

As used herein, the term “delayed release” refer to a type of modified release wherein a drug dosage form exhibits a time delay between oral administration of the drug dosage form and the release of the drug from said dosage form. Pulsed release systems (also known as pulsatile drug release”) and the use of enteric coatings, which are well known to those skilled in the art, are examples of delayed release mechanisms.

As used herein, the term “dissolution media” means aqueous solutions in which release of the drug from the tablet formulations is determined. These solutions could be potassium phosphate (monobasic) solutions with two concentrations (0.05M and 0.3M). 0.05 M and 0.3 M KH₂PO₄ represent high and low ionic strengths, respectively. pH of these solutions are adjusted to 6.0.

As used herein, the phrase “dissolution at a single pH”, “a single pH” or a “single pH system”, as used interchangeably herein, refers to the method described in Table 1 below:

TABLE 1

| Parameter | Condition | |
|---|---|---|
| Apparatus | USP Apparatus 2 (USP 29 NF 24) | |
| Agitation | 100 RPM ± 4% | |
| Medium | 1) | 0.05M potassium phosphate buffer 900 |
| | | mL, pH 6.0 ± 0.05 maintained at 37 ± |
| | | 0.5° C. |
| | 2) | 0.3M potassium phosphate buffer 900 |
| | | mL, pH 6.0 ± 0.05 maintained at 37 ± |
| | | 0.5° C. |

| | |
|---|---|
| Sampling Time Points | 30 minutes to 12 hours |
| UV Spectrophotometry | At 298 nm |
| Analysis | |

By an “effective amount” or a “therapeutically effective amount” of an active agent is meant a nontoxic but sufficient amount of the active agent to provide the desired effect. The amount of active agent that is “effective” will vary from subject to subject, depending on the age and general condition of the individual, the particular active agent or agents, and the like. Thus, it is not always possible to specify an exact “effective amount.” However, an appropriate “effective amount” in any individual case may be determined by one of ordinary skill in the art using routine experimentation.

As used herein, the term “extended release” or “sustained release” refers to a drug formulation that provides for gradual release of a drug over an extended period of time.

As used herein, a “fasted” patient, “fasting conditions” or “fasting” refers to a patient who has not eaten any food, i.e., who has fasted for at least 10 hours before the administration of the oral formulation of the present invention comprising at least one active agent and who does not eat any food and continues to fast for at least 4 hours after the administration of the formulation. The formulation is preferably administered with 240 ml of water during the fasting period, and water can be allowed ad libitum up to 1 hour before and 1 hour after ingestion.

As used herein, a “fed patient”, “fed conditions” or “fed” refers to a patient who has fasted for at least 10 hours overnight and then has consumed an entire test meal beginning 30 minutes before the first ingestion of the test formulations. The formulation of the present invention is administered with 240 ml of water within 5 minutes after completion of the meal. No food is then allowed for at least 4 hours post-dose. Water can be allowed ad libitum up to 1 hour before and 1 hour after ingestion. A high fat test meal provides approximately 1000 calories to the patient of which approximately 50% of the caloric content is derived from fat content of the meal. A representative high fat high calorie test meal comprises 2 eggs fried in butter, 2 strips of bacon, 2 slices of toast with butter, 4 ounces of hash brown potatoes and 8 ounces of whole milk to provide 150 protein calories, 250 carbohydrate calories and 500 to 600 fat calories. High fat meals can be used in clinical effect of food studies of fenofibric acid. A patient who receives such a high fat test meal is referred to herein as being under “high fat fed conditions”. A low fat test meal provides approximately 500 calories to the patient of which approximately 30% of the caloric content is derived from fat content of the meal. A patient who receives such a low fat test meal is referred to herein as being under “low fat fed conditions”.

As used herein, the terms “formulation”, “form” or “dosage form” as used interchangeably herein, denotes any form of a pharmaceutical composition that contains an amount of active agent sufficient to achieve the desired therapeutic effect. The frequency of administration that will provide the most effective results in an efficient manner without overdosing will vary with the characteristics of the particular active agent, including both its pharmacological characteristics and its physical characteristics.

As used herein, the term “hydrophilic polymer” include, but are not limited to, hydroxypropyl methylcellulose (HPMC), hydroxypropyl cellulose, hydroxyethyl cellulose, polyethylene oxide, polyethylene glycols (“PEG”), xanthum gum, alginates, polyvinyl pyrrolidone, starches, cross-linked homopolymers and copolymers of acrylic acid and other pharmaceutically acceptable substances with swelling and/or gel-forming properties and combinations thereof.

As used herein, the term “ionic strength” of a solution means concentration of ions in a solution or a function of the concentration of ions in a solution. It can be calculated based on the molality of the concentration of ions and the charges of ions.

As used herein, the term “IDR” is abbreviation of intrinsic dissolution rate. The intrinsic dissolution rate is the rate of dissolution of pharmaceutically acceptable ingredients when conditions such as surface area, agitation or stirring speed, pH and ionic strength of the dissolution medium are held constant.

As used herein, the term “inert substrate” refers to (a) water insoluble substrates or seeds comprising different oxides, celluloses, organic polymers and other materials, alone or in mixtures; or (b) water soluble substrates or seeds comprising different inorganic salts, sugars, non-pareils and other materials, alone or in mixtures.

As used herein, the term “membrane” refers to a film or layer that is permeable to aqueous solutions or bodily fluids and may also be permeable to the active agent.

As used herein, the term “modified” refers to a drug containing formulation in which release of the drug is not immediate (See, for example, Guidance for Industry SUPAC-MR: Modified Release Solid Oral Dosage Forms, Scale-Up and Postapproval Changes: Chemistry, Manufacturing, and Controls; In Vitro Dissolution, Testing and In Vivo Bioequivalence Documentation, U.S. Department of Health and Human services, Food and Drug Administration, Center for Drug Evaluation and Research (“CDER”), September 1997 CMC 8, page 34, herein incorporated by reference.). In a modified formulation, administration of said formulation does not result in immediate release of the drug or active agent into an absorption pool. The term is used interchangeably with “nonimmediate release” as defined in Remington: The Science and Practice of Pharmacy, Nineteenth Ed. (Easton, Pa.: Mack Publishing Company, 1995). As used herein, the term “modified release” includes extended release, sustained release, delayed release, and controlled release formulations.

As used herein, the phrase “pharmaceutically acceptable,” such as in the recitation of a “pharmaceutically acceptable excipient,” or a “pharmaceutically acceptable additive,” is meant a material that is non-toxic or otherwise physiologically acceptable.

As used herein, the term “soluble salt” means all feno acid salts of which the solubility in water at 25° C. is greater than 16.1 mg/ml.

As used herein, the term “subject” refers to an animal, preferably a mammal, including a human or non-human. The terms patient and subject may be used interchangeably herein.

As used herein the term “substantially independent” of ionic strength means release of the drug, fenofibric acid salts, from the tablet formulations in the dissolution media is less affected by the change in ionic strength of the dissolution media, that is, the difference in % drug released when dissolutions are conducted in media of low (0.05M) and high (0.3M) ionic strengths at each time point within 8 hours is less 25%.

As used herein, the terms “treating” and “treatment” refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

I. Salt Selection

Dissolution Rates and Disintegration Times

Applicants have determined that the selection of the salt in a fenobric acid salt formulation affects the robustness of the formulation. Applicants studied the release rates of fenofibric acid formulations comprising seven different salts of fenofibric acid and fenofibric acid alone. The ingredients for each of the studied formulations are shown in Table 2. The method used to make the tablets is described in Example 1, which follows Table 2.

The solubility of each salt was determined according to Example 2. Likewise, the IDR values for each salt of fenofibric acid were determined according to Example 3. The salts of fenofibric acid and their respective solubility and IDR are shown in Table 4.

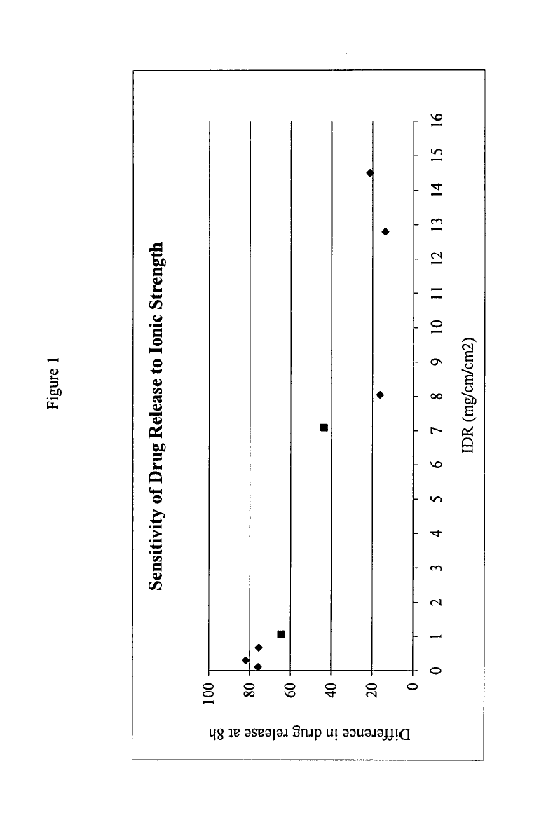

Applicants determined the dissolution rates of each of the fenofibric acid salt formulations in dissolution media at a high and low ionic strength using the single pH method as defined above. Table 4 shows the % dissolved after 8 hours at 0.05M and 0.3M and the difference for each formulation at these ionic strengths. Applicants have depicted their findings in FIG. 1. The graph in FIG. 1 plots the IDR for each fenofibric acid salt formulation verses the difference in dissolution values at 8 hours. As can be seen in FIG. 1 and in Table 4 the fenofibric acid salts with greater salt solubility and higher IDR values are less sensitive to the ionic strength of the dissolution media (that is the difference in the dissolution values at 8 hours and throughout the profile is less when compared at high and low ionic strengths).

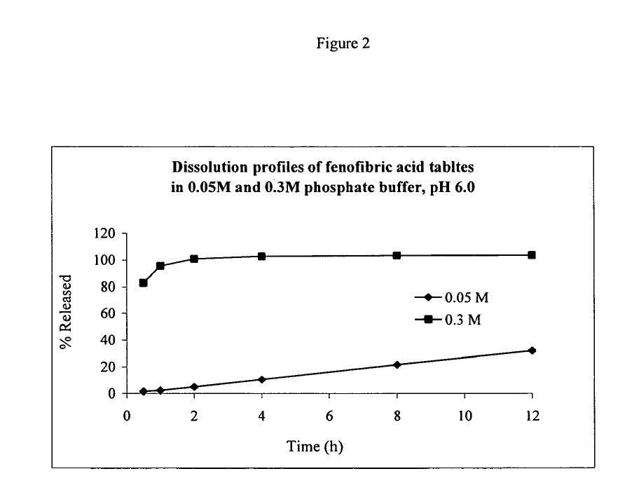

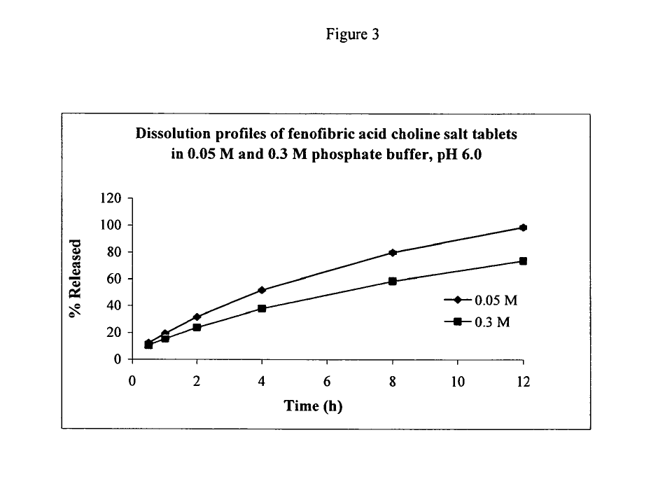

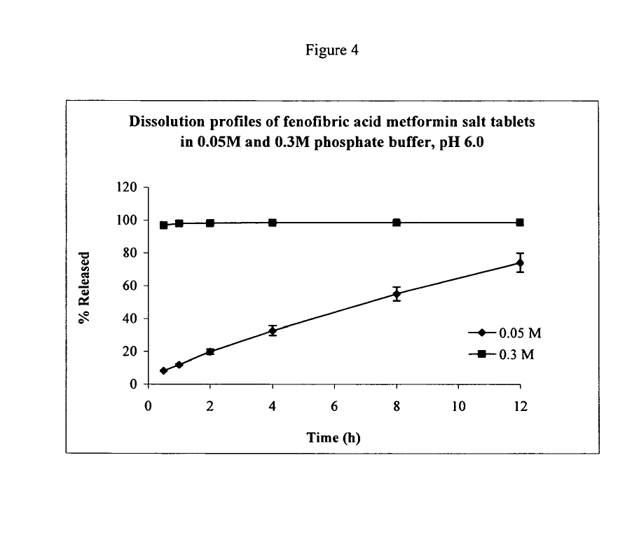

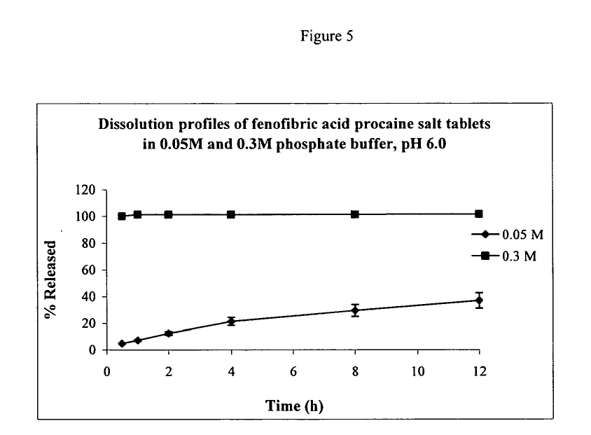

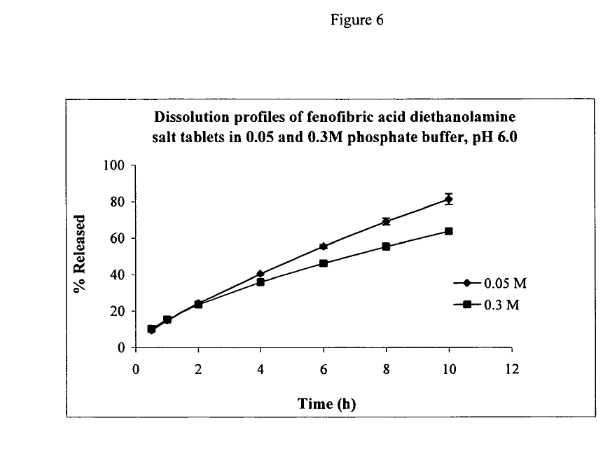

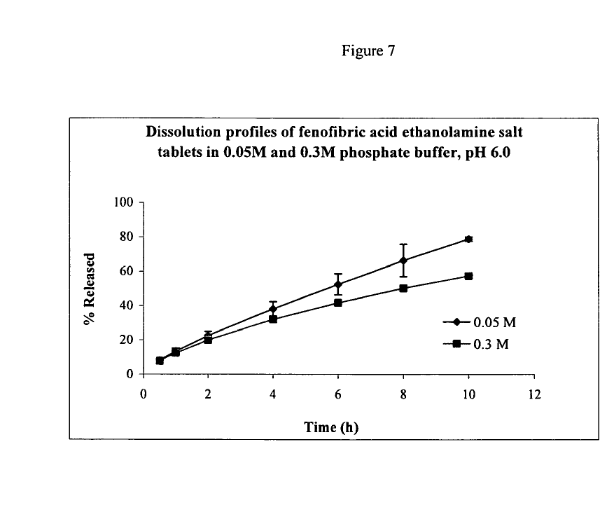

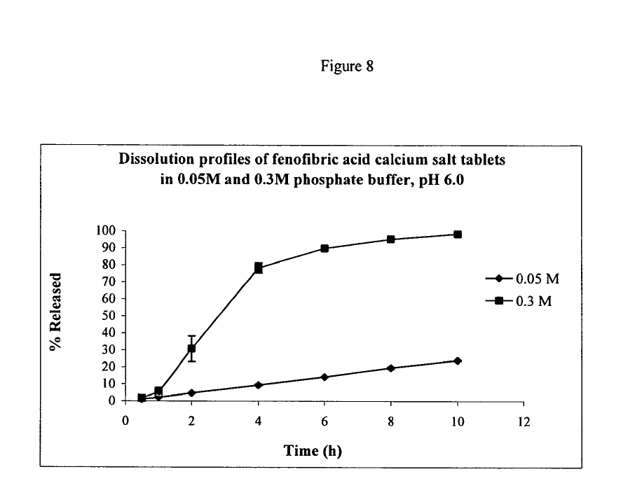

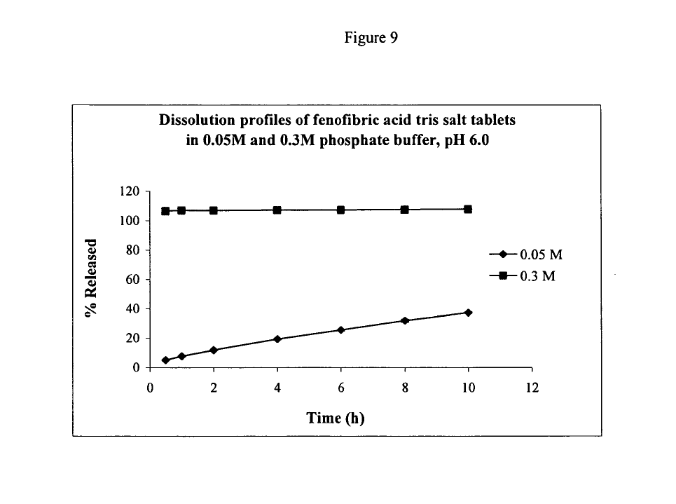

FIGS. 2-9 show the dissolution profiles for the fenofibric acid salt and fenofibric acid formulations at 0.05M and 0.3M ionic strength dissolution media (Table 5 shows dissolution data for formulations tested in media of low ionic strength and Table 6 shows the dissolution data for formulations tested in media of high ionic strength). As can be seen from these figures the formulations with the more soluble fenofibric acid salts are more robust and thus the release rates are less sensitive to the ionic strength of the dissolution media.

TABLE 2

| | Formulations (%) | | | | | | | |
|---|---|---|---|---|---|---|---|---|
| Ingredient | A | B | C | D | E | F | G | H |
| Intragranular | | | | | | | | |
| Feno acid | 65.5 | | | | | | | |
| Choline salt | | 65.5 | | | | | | |
| Diethanolamine | | | 65.5 | | | | | |
| salt | | | | | | | | |
| Ethanolamine | | | | 65.5 | | | | |
| salt | | | | | | | | |
| Metformin salt | | | | | 65.5 | | | |
| Procaine salt | | | | | | 65.5 | | |
| Tris salt | | | | | | | 65.5 | |
| Calcium salt | | | | | | | | 65.5 |
| HPMC K15M | 27 | 27 | 27 | 27 | 27 | 27 | 27 | 27 |
| PVP K30 | 3 | 3 | 3 | 3 | 3 | 3 | 3 | 3 |
| Water | qs | qs | qs | qs | qs | qs | qs | qs |
| Extragranular | | | | | | | | |
| Silicon dioxide | 0.5 | 0.5 | 0.5 | 0.5 | 0.5 | 0.5 | 0.5 | 0.5 |
| HPC exf | 3 | 3 | 3 | 3 | 3 | 3 | 3 | 3 |
| SSF | 1 | 1 | 1 | 1 | 1 | 1 | 1 | 1 |

Tablet weight: 275 mg

### Example 1

### Tablet Preparation

The intra granular ingredients were added into a granulator (or mixer) and dry mixed followed by gradual addition of a suitable amount of water to the granulator and granulating until optimal granulation was achieved. The granulation was then wet massed if necessary for an additional period of time and then dried in an oven or a fluid bed dryer. The dried granules were using the fitzmill or manually screened using a mesh. The Silicon Dioxide and HPC Exf were screened through a 40-mesh screen. The milled granules, and screened silicon dioxide and HPC were charged into a V-blender and blended for 5 minutes at ˜26 rpm. The SSF was screened through a 40-mesh screen. The screened SSF was added into the blender and blended for additional 5 minutes. The granules were weighed and compressed using the rounder tooling into a table with target weight of 275 mg/tablet. Target tablet hardness was ˜20 SCU.

### Example 2

Solubility Determination: Solubility values of fenofibric acid salts in water were determined at 25° C. The salts were weighed into glass vials and water was added. The suspensions were rotated from end to end for about 2 days in a 25° C. water bath. The pH of the suspensions was measured. The residual solid was then removed via filtration through a 0.45 μm PTFE membrane filter. The resulting saturated solution was diluted appropriately into the HPLC mobile phase, and analyzed by the HPLC assay described below (Table 3). The powder x-ray diffraction pattern of the collected residual solid was recorded at the end of experiment.

### HPLC Analysis:

TABLE 3
HPLC Assay for Fenofibric Acid.

| Parameters | Conditions |
|---|---|
| Column | Waters Symmetry Shield ®, RP18, |
| | 5 μm, 250 × 4.6 mm |
| Autosampler Temperature | Ambient |
| Column Temperature | ~35° C. |
| Flow Rate | ~1 ml/min |
| Detection Wavelength | 286 nm |
| Injection Volume | 25 μl |
| Mobile phase A | 25 mM K₂HPO₄ in water, pH adjusted |
| | to 2.5 with H₃PO₄ |
| Mobile phase B | Acetonitrile |
| Isocratic elution | A/B = 40/60 |
| Retention time | ~8 minutes |

### Example 3

### Intrinsic Dissolution Rate (IDR)

The IDR of salts of fenfibric acid were determined in 50 mM sodium citrate buffer at pH 4.0 or pH 6.8 (μ=0.155 M with NaCl).

Pellets of the salts were prepared by compressing ca. 100 mg of the compound in a stainless steel die under 1300 pounds force with a dwell time of one minute. The die containing the tablet was submerged in 400 mL of the dissolution medium at 37° C. The solution was stirred by a paddle at =60 rpm. At each time point, 3 mL of sample was withdrawn and filtered. After discarding the first half of the filtrate, the remainder was collected and assayed by HPLC method above. The total volume of the dissolution medium was kept at a constant by replenishing the lost volume at each data point with fresh buffer at 37 C.

TABLE 4

| Salt | Solubility (mg/ml) | IDR* | % in 0.05M@8 h | % in 0.3M@8 h | Difference@8 h |
|---|---|---|---|---|---|
| Choline | >300 | 14.50 | 80.0 | 58.6 | 21.4 |
| Diethanolamine | >250 | 12.80 | 69.2 | 55.5 | 13.7 |
| Ethanolamine | 19.0 | 8.05 | 66.3 | 50.1 | 16.2 |
| Metformin | 16.1 | 7.09 | 55.2 | 98.8 | 43.6 |
| Procaine | 7.2 | 1.06 | 37.0 | 101.6 | 64.6 |
| Tris | 5.45 | 0.67 | 32.0 | 107.5 | 75.5 |
| Calcium | 0.36 | 0.10 | 19.6 | 95.3 | 75.7 |
| Free acid | 0.265 | 0.30 | 21.6 | 103.5 | 81.9 |

*units for IDR mg/min/cm²; IDR measured at a pH of 6.8

TABLE 5

| | Dissolution of Formulations in 0.05M Phosphate Buffer, pH 6.0 (65.5% Loading) | | | | | | | |
|---|---|---|---|---|---|---|---|---|
| Time | A | B | C | D | E | F | G | H |
| (h) | Free acid | Choline | Diethanolamine | Ethanolamine | Metformin | Procaine | Tris | Calcium |
| 0.5 | 1.6 | 12.2 | 9.5 | 8.0 | 8.3 | 4.9 | 5.2 | 1.1 |
| 1 | 2.4 | 19.3 | 15.1 | 13.3 | 12 | 7.3 | 7.8 | 2.2 |
| 2 | 5 | 31.8 | 24.3 | 22.5 | 19.8 | 12.4 | 12.0 | 4.7 |
| 4 | 10.5 | 51.8 | 40.5 | 38.1 | 32.8 | 21.4 | 19.4 | 9.4 |
| 6 | | | 55.6 | 52.4 | 44.3 | 29.5 | 25.6 | 14.3 |
| 8 | 21.6 | 80 | 69.2 | 66.3 | 55.2 | 37.0 | 32.0 | 19.6 |
| 10 | | | 81.4 | 78.9 | 65 | 43.9 | 37.4 | 24.0 |
| 12 | 32.1 | 98.7 | | | 74.3 | 50.5 | | |

TABLE 6

| | Dissolution of Formulations in 0.3M Phosphate Buffer pH 6.0 (65.5% Loading) | | | | | | | |
|---|---|---|---|---|---|---|---|---|
| Time | A | B | C | D | E | F | G | H |
| (h) | Free acid | Choline | Diethanolamine | Ethanolamine | Metformin | Procaine | Tris | Calcium |
| 0.5 | 82.9 | 10.6 | 10.4 | 7.8 | 97 | 100.2 | 106.5 | 1.7 |
| 1 | 95.6 | 15.3 | 15.6 | 12.3 | 97.9 | 101.4 | 107.0 | 5.3 |
| 2 | 101 | 23.8 | 23.6 | 19.9 | 98.2 | 101.4 | 107.0 | 30.8 |
| 4 | 102.9 | 37.9 | 36.0 | 32.0 | 98.5 | 101.4 | 107.2 | 78.3 |
| 6 | | | 46.3 | 41.8 | 98.6 | 101.4 | 107.3 | 89.9 |
| 8 | 103.5 | 58.6 | 55.5 | 50.1 | 98.7 | 101.6 | 107.5 | 95.3 |
| 10 | | | 63.8 | 57.3 | 98.8 | 101.8 | 107.7 | 98.4 |
| 12 | 103.8 | 73.7 | | | 98.9 | 101.7 | | |

Applicants also measured the disintegration times of fenofibric acid salt formulations and determined that the more soluble the salt the less disintegration time would be impacted by the ionic strength of the media. The method for measuring disintegration time is presented in Example 4. The disintegration times for the choline fenofibric acid salt, the diethanolamine fenofibric acid salt and fenofibric acid are presented in Table 7.

### Example 4

### Disintegration

Disintegration times were determined by dropping tablets into a heated (37° C.) aqueous media (900 ml 0.05M KH₂PO₄ pH 6.0 and 900 ml 0.3M KH₂PO₄ pH 6.0). The tablets were then bobbed up and down at a fixed rate until they were fully disintegrated, the time for disintegration was recorded in minutes.

TABLE 7

| | Disintegration Time (minutes) | | |
|---|---|---|---|
| Disintegration | | Diethanol- | |
| Medium pH 6.0 | Choline (B) | amine (C) | Fenofibric acid (A) |
| .3M phosphate | 47 ± 5 | 39 ± 3.6 | 11 ± 1 |
| buffer | | | |
| .05M phosphate | 66 ± 1 | 71 ± 12 | 486 ± 17 |
| buffer | | | |
| Difference | 19 | 32 | 475 |
| (minutes) | | | |

II. Salt of Fenofibric Acid Concentration

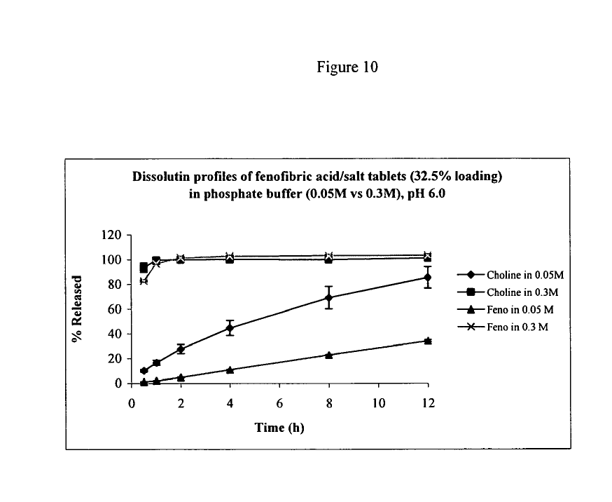

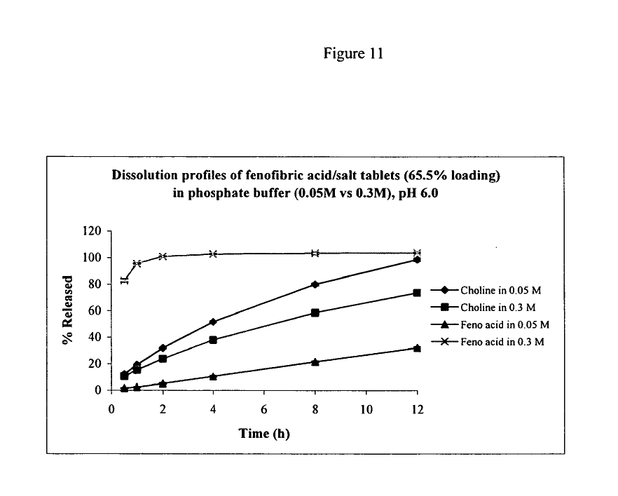

Applicants have discovered that the percentage of the fenofibric acid salt in the formulation also impacts the robustness of the formulation. Applicants compared formulations with different percentages of fenofibric acid salt or fenofibric acid and found that when the percentage of the fenofibric acid salt or fenofibric acid is between 33 and 75 the formulation is most robust. Applicants compared the robustness of formulations I and K (presented in Table 8) to formulations A and B (presented in Table 2) by evaluating the impact of the ionic strength of the dissolution media on the dissolution rate of the formulation. FIGS. 10 and 11 depict the dissolution curves for the formulations of different concentration active ingredient. FIG. 10 shows the release rate of formulations I and K with 32.5% drug load and FIG. 11 shows the release profile of formulations A and B at 65.5% drug load in dissolution media of high and low ionic strengths. Applicants discovered that the dissolution profiles of fenofibric acid salt formulations are less affected by the ionic strength at a higher drug load.

TABLE 8

Formulations (%)

| Ingredient | I | J | K | L |
|---|---|---|---|---|
| Intragranular | | | | |
| Feno acid | | 49.5 | 32.75 | |
| Feno Choline salt | 32.75 | | | |
| HPMC K15M | 27 | 27 | 27 | 27 |
| PVP K30 | 3 | 3 | 3 | 3 |
| Lactose monohydrate | 32.75 | 16 | 32.75 | 65.5 |
| Water | qs | qs | qs | qs |
| Extragranular | | | | |
| Silicon dioxide | 0.5 | 0.5 | 0.5 | 0.5 |
| HPC exf | 3 | 3 | 3 | 3 |
| SSF | 1 | 1 | 1 | 1 |

Tablet weight: 275 mg

III. Enteric Coating

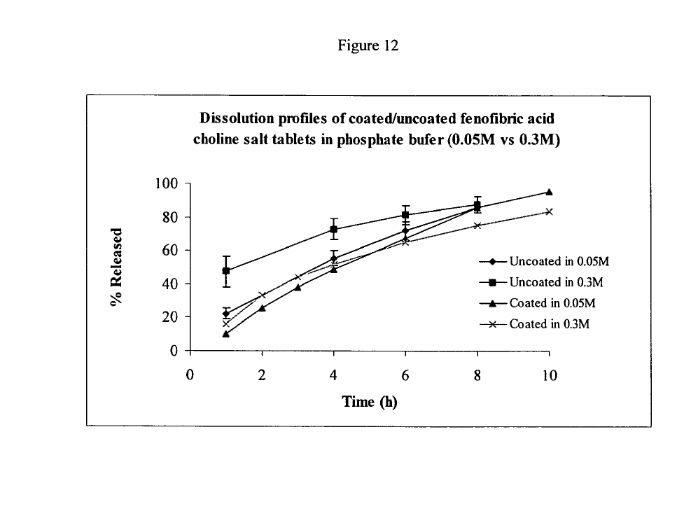

Applicants have determined that the presence or absence of an enteric coating may have some influence on the robustness of the formulation. Applicants compared the dissolutions profiles of fenofibric acid choline salt made with and without a coating. The composition of the formulations tested, with and without the coating, is shown in Table 9. These tablets were manufactured according to the manufacturing process of Example 6. FIG. 12 shows the dissolution profiles of the coated and uncoated tablets when dissolved in the 0.05 M and 0.3 M dissolution media. As shown in FIG. 12, the coated tablets' dissolution is less impacted by the ionic strength of the dissolution media.

TABLE 9

| | Fenofibric acid |
|---|---|
| | choline salt (with or |
| | without coating) |
| Intra-granule | |
| Fenofibric Acid Choline Salt | 65 |
| HPMC K15M | 15 |
| Avicel PH101 | 15.75 |
| PVP K30 | 3.0 |
| Extra-granules | |
| Silicon Dioxide | 0.75 |
| Magnesium Stearate | 0.5 |
| Coating (optional) | |
| Eudragit L30 D55 | 10.61 |
| Talc | 5.31 |
| Triethyl Citrate | 1.59 |

### Example 5

### Manufacturing Process for Coated and Uncoated Tablets

Granulations were prepared by dry blending the powders, followed by the gradual addition of water until optimal granulation was achieved. The granulation was then wet massed if necessary for an additional period of time and then dried in an oven or a fluid bed dryer. The dried granulation was milled using the fitzmill or manually screened using a mesh and then blended with the extra-granular excipients such as magnesium stearate. The final blend was weighed out and punched into tablets using a compression machine. Tablets were optionally coated using a pan coater.

One skilled in the art would readily appreciate that the present invention is well adapted to carry out the objects and obtain the ends and advantages mentioned, as well as those inherent therein. The compositions, formulations, methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1-13. (canceled)

14. A method of treating hyperlipidemia, hypercholesterolemia, or hypertriglyceridemia comprising the step of orally administering a pharmaceutical composition to a human subject in need thereof, wherein the pharmaceutical composition comprises:
(a) a salt of fenofibric acid, wherein the process for selecting said salt of fenofibric acid comprises the steps of:
(i) creating a modified-release oral dosage form comprising:
(1) a salt of fenofibric acid;
(2) a hydrophilic polymer; and
(3) optionally, one or more pharmaceutically acceptable excipients; and
(ii) choosing a dosage form of step (i) having at least one of the following properties:
(1) the release rate of fenofibric acid from the dosage form is substantially independent of the ionic strength of the dissolution media;
(2) the difference between the amount of fenofibric acid salt dissolved at 0.5, 1, 2, 4, 6, or 8 hours in (A) 900 mL of 0.05 M potassium phosphate buffer at a pH of 6.0 and a temperature of 37° C. and (B) 900 mL of 0.3 M potassium phosphate buffer at a pH of 6.0 and a temperature of 37° C., is not greater than about 25%; or
(3) the difference between disintegration times in (A) 900 mL of 0.05 M potassium phosphate buffer at a pH of 6.0 and a temperature of 37° C. and (B) 900 mL of 0.3 M potassium phosphate buffer at a pH of 6.0 and a temperature of 37° C., is less than about 475 minutes; and
(b) a hydrophilic polymer.

15. The method of claim 14, wherein the salt of fenofibric acid has an aqueous solubility of greater than about 16.1 mg/mL.

16. The method of claim 14, wherein the salt of fenofibric acid has an aqueous solubility of greater than about 19.0 mg/mL.

17. The method of claim 14, wherein the salt of fenofibric acid has an intrinsic dissolution rate of greater than about 7.09 mg/min/cm2 in 400 mL of a 50 mM sodium citrate buffer at a pH of 6.8.

18. The method of claim 14, wherein the salt of fenofibric acid has an intrinsic dissolution rate of greater than about 8.05 mg/min/cm2 in 400 mL of a 50 mM sodium citrate buffer at a pH of 6.8.

19. The method of claim 14, wherein the difference in (a)(ii)(2) between the amount of fenofibric acid salt dissolved at 0.5, 1, 2, 4, 6, or 8 hours in (A) 900 mL of 0.05 M potassium phosphate buffer at a pH of 6.0 and a temperature of 37° C. and (B) 900 mL of 0.3 M potassium phosphate buffer at a pH of 6.0 and a temperature of 37° C., is not greater than about 21.4%.

20. The method of claim 14, wherein the difference in (a)(ii)(3) between disintegration times in (A) 900 mL of 0.05 M potassium phosphate buffer at a pH of 6.0 and a temperature of 37° C. and (B) 900 mL of 0.3 M potassium phosphate buffer at a pH of 6.0 and a temperature of 37° C., is less than about 100 minutes.

20. The method of claim 14, wherein the difference in (a)(ii)(3) between disintegration times in (A) 900 mL of 0.05 M potassium phosphate buffer at a pH of 6.0 and a temperature of 37° C. and (B) 900 mL of 0.3 M potassium phosphate buffer at a pH of 6.0 and a temperature of 37° C., is less than about 100 minutes.

21. The method of claim 14, wherein the hydrophilic polymer is selected from the group consisting of: hydroxypropylmethylcellulose, hydroxypropylcellulose, hydroxyethylcellulose, polyethylene oxide, polyethylene glycols, xanthum gum, alginates, polyvinylpyrrolidone, starches, cross-linked homopolymers, and copolymers of acrylic acid.

22. The method of claim 14, wherein the hydrophilic polymer is hydroxypropylmethylcellulose.

23. The method of claim 14, wherein the salt of fenofibric acid is present in an amount of between about 33% and about 75% by weight of the formulation.

24. The process of claim 14, wherein the salt of fenofibric acid is present in an amount of between about 50% and about 75% by weight of the formulation.

25. The process of claim 14, wherein the salt of fenofibric acid is present in an amount of about 65.5% by weight of the formulation.

26. A method of treating hyperlipidemia, hypercholesterolemia, or hypertriglyceridemia comprising the steps of:
(a) creating a modified-release pharmaceutical dosage form suitable for oral administration to a human subject comprising:
(i) a salt of fenofibric acid;
(ii) a hydrophilic polymer; and
(iii) optionally, other pharmaceutically acceptable excipients; and wherein the pharmaceutical dosage form has at least one of the following properties:
(1) the release rate of fenofibric acid from the dosage form is substantially independent of the ionic strength of the dissolution media;
(2) the difference between the amount of fenofibric acid salt dissolved at 0.5, 1, 2, 4, 6, or 8 hours in (A) 900 mL of 0.05 M potassium phosphate buffer at a pH of 6.0 and a temperature of 37° C. and (B) 900 mL of 0.3 M potassium phosphate buffer at a pH of 6.0 and a temperature of 37° C., is not greater than about 25%; or
(3) the difference between disintegration times in (A) 900 mL of 0.05 M potassium phosphate buffer at a pH of 6.0 and a temperature of 37° C. and (B) 900 mL of 0.3 M potassium phosphate buffer at a pH of 6.0 and a temperature of 37° C., is less than about 475 minutes; and (b) orally administering the pharmaceutical dosage form to a human subject in need thereof.

27. The method of claim 26, wherein the salt of fenofibric acid has an aqueous solubility of greater than about 16.1 mg/mL.

28. The method of claim 26, wherein the salt of fenofibric acid has an intrinsic dissolution rate of greater than about 7.09 mg/min/cm2 in 400 mL of a 50 mM sodium citrate buffer at a pH of 6.8.

29. The method of claim 26, wherein the difference in (a)(2) between the amount of fenofibric acid salt dissolved at 0.5, 1, 2, 4, 6, or 8 hours in (A) 900 mL of 0.05 M potassium phosphate buffer at a pH of 6.0 and a temperature of 37° C. and (B) 900 mL of 0.3 M potassium phosphate buffer at a pH of 6.0 and a temperature of 37° C., is not greater than about 21.4%.

30. The method of claim 26, wherein the difference in (a)(3) between disintegration times in (A) 900 mL of 0.05 M potassium phosphate buffer at a pH of 6.0 and a temperature of 37° C. and (B) 900 mL of 0.3 M potassium phosphate buffer at a pH of 6.0 and a temperature of 37° C., is less than about 100 minutes.
